(19) United States
(12) Patent Application Publication
(10) Pub. No.: US 2022/0079167 A1
(43) Pub. Date: Mar. 17, 2022

(54) TOPICAL MOSQUITO CONTROL PRODUCT WITH SUNSCREEN

(71) Applicant: Pouch Pac Innovations, LLC, Sarasota, FL (US)

(72) Inventors: R. Charles Murray, Sarasota, FL (US); John Harlin, Sarasota, FL (US)

(21) Appl. No.: 17/531,925

(22) Filed: Nov. 22, 2021

Related U.S. Application Data
(63) Continuation-in-part of application No. 16/554,843, filed on Aug. 29, 2019.
Application No. 16/554,843 is a continuation-in-part of application No. 15/656,676, filed on Jul. 21, 2017.
Application No. 15/656,676 is a continuation-in-part of application No. 15/412,568, filed on Jan. 23, 2017, now abandoned.
(60) Provisional application No. 63/116,295, filed on Nov. 20, 2020.
(60) Provisional application No. 62/416,399, filed on Nov. 2, 2016.
(60) Provisional application No. 62/281,369, filed on Jan. 21, 2016.

(51) Int. Cl. A01N 65/28; A61K 8/27; A61K 8/92; A61K 8/36; A61K 8/55; A61K 8/35; A61K 8/37; A01N 65/22; A01N 65/20; A61Q 17/04; A01P 17/00

(52) U.S. Cl. CPC A01N 65/28; A61K 8/27; A61K 8/922; A61K 8/361; A61K 8/553; A61K 8/35; A61K 2800/412; A61K 8/927; A01N 65/22; A01N 65/20; A61Q 17/04; A01P 17/00; A61K 8/37

(57) ABSTRACT

A topical mosquito repellent and sunscreen product includes active and inert ingredients. The active ingredients include natural materials and may include soybean oil, clove oil, thyme oil and geraniol and the inert ingredients include bee [symbol] s wax, sunflower oil, steric acid, zinc oxide, water, vanillin, lecithin, and triethyl citrate.

### CROSS-REFERENCE TO RELATED APPLICATIONS

The instant application is a CIP application of U.S. application Ser. No. 15/656,676 filed on Jul. 21, 2017 which is a CIP application of U.S. application Ser. No. 15/412,568 filed on Jan. 23, 2017 which claims priority to U.S. Provisional Application Ser. No. 62/281,369 filed Jan. 21, 2016 and U.S. Provisional Application Ser. No. 62/416,399 filed Nov. 2, 2016 the entire contents of which are incorporated herein by reference.

### FIELD OF THE INVENTION

The instant application is directed to a formulation for a topical mosquito repellent with sunscreen.

### BACKGROUND OF THE INVENTION

Mosquito bites often cause annoying reactions in humans and animals. Additionally, mosquito bites are a vector for many types of disease including: malaria, Dengue Fever, Chikungunya, West Nile and Zika viruses as well as other forms of disease.

There is a need to prevent mosquitos from biting a host to lessen the discomfort of a bite reaction and to prevent the potential spread of a disease.

Accordingly, a natural non-toxic ingredient based topical product that prevents bites to humans and animals and includes sunscreen protection would be desirable.

### SUMMARY OF THE INVENTION

In one aspect, there is disclosed a topical mosquito repellent and sunscreen composition including a lotion having active and inert ingredients. The active ingredients include soybean oil, clove oil, thyme oil and geraniol and the inert ingredients include bee [symbol] s wax, sunflower oil, steric acid, zinc oxide, water, vanillin, lecithin, and triethyl citrate.

Also disclosed is a method of repelling insects which includes applying the lotion compositions to a body and exposing insects to the lotion. Additionally, the lotion composition includes an SPF factor of greater than 30.

Additional features and advantages of mosquito control products described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein and together with the description serve to explain the principles and operations of the claimed subject matter.

### BRIEF DESCRIPTION OF THE DRAWINGS

FIG. 1 is a graphical representation of an experimental apparatus used for testing;

FIG. 2 is a graphical depiction of an experimental apparatus positioned over a blood source;

FIG. 3 is a graphical representation of an experimental apparatus including a circulating pump providing warm water to the testing apparatus;

FIG. 4 is a report for SPF testing;

FIG. 5 is a graphical depiction of an experimental apparatus positioned over a blood source;

FIG. 6 is a graphical depiction of an experimental apparatus and application of repellents;

FIG. 7 is a graphical depiction of the temperature and humidity readings of example 4;

FIG. 8 is a graph of the mean bite count as a function of time for repellents of example 4;

FIG. 9 is a graph of the % repellency as a function of time for repellents of example 4.

### DETAILED DESCRIPTION OF THE EMBODIMENTS

Mosquito bites may transmit diseases and cause reactions to people that are bitten. Various repellant compositions have been utilized in prior art repellant compositions. One such repellant, DEET, is often used in mosquito repellant compositions. DEET has been questioned as to its toxicity in higher amounts for use with humans and other subjects. There is therefore a need in the art for an improved and safe topical mosquito repellant composition.

In one aspect, there is disclosed herein a formulation that may be utilized as a topical lotion or spray to repel mosquitos and provide sunscreen protection. The formulation may include organic and chemical-free compositions that will protect humans and other animals from blood-seeking insects such as mosquitos as well as provide sunscreen protection.

In one aspect the composition may include bee [symbol] s wax, soybean oil, edible oil such as avocado oil or sunflower oil, isopropyl alcohol, peppermint oil, citronella oil, thyme oil, vanillin, water, zinc oxide. Lecithin and turmeric oil. Various amounts of these materials may be present in the composition as will be described in more detail below.

As described above, the composition may include active ingredients and inactive ingredients. The soybean oil, citronella oil, peppermint oil and thyme oil may be classified as active ingredients wherein the bee [symbol] s wax, edible oil such as avocado oil or sunflower oil, isopropyl alcohol, vanillin, water, zinc oxide, lecithin and turmeric oil may be considered inert or inactive ingredients.

### Example 1

In one aspect, the lotion formulation may include a sunscreen component with a SPF value of 30. The formulation may include:

| | | | ml (+/− | Weight |
|---|---|---|---|---|
| Ingredient | grams | x density | 1%) | Percentage |
| Avocado Oil | 390 | 0.915 | 356.85 | 39% |
| Soy Bean Oil | 330 | 0.917 | 302.61 | 33% |
| Beeswax | 200 | 0.96 | 192 | 20% |
| Isopropyl | 65 | 0.7854 | 51 | 6.50% |
| Citronella Oil | 3 | 0.855 | 2.57 | 0.30% |
| Peppermint Oil | 4 | 0.905 | 3.62 | 0.40% |
| Thyme Oil | 1.5 | 0.941 | 1.41 | 0.15% |
| Vanillin | 1.5 | 1.056 | 1.58 | 0.15% |
| (powder) | | | | |
| Water | 5 | 1 | 5 | 0.50% |
| Total | 1000 | | 916.64 | 100% |

As specified above, the composition may include bee [symbol] s wax, soybean oil, edible oil such as avocado oil, isopropyl alcohol, peppermint oil, citronella oil, and thyme oil. The bee [symbol] s wax may be present in an amount of from 15 to 25% by weight. The soybean oil may be present in an amount of from 25 to 40% by weight. The avocado oil may be present in an amount of from 35 to 50% by weight. The isopropyl alcohol may be present from 5 to 15% by weight. The peppermint oil may be present at about 1% by weight. The citronella oil may also be present in an amount of from 0.3 to 0.5% by weight. The thyme oil may be present at about 0.1 to 0.20% by weight. Additionally, the composition may include powdered ingredients such as vanillin present at about 0.1 to 0.20% by weight. The weight percentages are based on a total weight of the composition.

The formulation includes approximately 80% oils and liquid to beeswax, however this ratio can be altered by increasing/or decreasing beeswax by 3 to 5% for a desired consistency for varied temperature environments. The density of product as formulated above is 0.917 g/ml. The active ingredients in the above formulation may include: soybean oil, citronella oil, peppermint oil, and thyme oil. The avocado oil, bee [symbol] s wax, isopropyl alcohol, vanillin and water may be inert ingredients.

The composition of example 1 may be formed by heating the soy oil, avocado oil to approximately 140° F. Next, the beeswax is added to the composition. Next, the citronella oil, peppermint oil, thyme oil are added to the composition and mixed thoroughly. The composition is then allowed to cool to room temperature forming a stiff mass. Next, the composition is blended with a lotion substrate to form a cream lotion or spray. Various ratios of the composition and substrate may be utilized, for example a ratio of 50 to 4, 48 to 6, and 50 to 8 may be utilized with respect to the composition and the substrate cream component.

### Example 2

In another aspect, the lotion and sunscreen formulation may include:

| Ingredient | Density | Weight percentage |
|---|---|---|
| Avocado Oil | .915 | 44% |
| Soy Bean Oil | .917 | 26% |
| Beeswax | .960 | 16% |
| Zinc Oxide | 5.61 | 6% |
| Isopropyl | .7854 | 5% |
| Citronella Oil | .855 | 1% |
| Pepper Mint Oil | .905 | 1% |
| Thyme Oil | .941 | 0.5% |
| Vanillin (powder) | 1.056 | 0.4% |
| Lecithin ( powder) | 1.030 | 0.1% |

As specified above, the composition may include bee [symbol] s wax, soybean oil, edible oil such as avocado oil, isopropyl alcohol, peppermint oil, citronella oil, and thyme oil. The bee [symbol] s wax may be present in an amount of from 10 to 20% by weight. The soybean oil may be present in an amount of from 20 to 30% by weight. The avocado oil may be present in an amount of from 35 to 50% by weight. The isopropyl alcohol may be present from 5 to 15% by weight. The peppermint oil may be present at about 1% by weight. The citronella oil may also be present in an amount of from 1% to 2% by weight. The thyme oil may be present at about 0.5% by weight. Additionally, the composition may include powdered ingredients such as vanillin present at about 0.4% by weight and Lecithin present at about 0.1% by weight and zinc oxide at about 6% by weight. The weight percentages are based on a total weight of the composition.

The active ingredients in the above formulation may include: soybean oil, citronella oil, peppermint oil, and thyme oil. The bee [symbol] s wax, avocado oil, isopropyl alcohol, vanillin, water, zinc oxide and lecithin may be inert ingredients.

The composition of example 2 was made according to the following procedure. The soybean oil and avocado oils were heated from room temperature to 145 to 160 degrees F. in a mixing vat under agitation. The vanillin, lecithin and bee [symbol] s wax were added to the composition when the temperature approaches 100 F. The mixture is heated and agitated until the mixture clears. Once the mixture clears heat is removed from the composition. At Temperature of 145 F the zinc oxide is added and shearing of the mixture begins. Water is then added to the composition under agitation and the composition is cooled to 110 F. Isopropyl alcohol is added at the temperature of 110 F. The mixture immediately cools to 100 F. At this time the peppermint oil, citronella oil and thyme oil are added to the composition. The composition is agitated and sheared for 30 minutes. After which time the lotion may be packaged.

### Example 3

In a further aspect, the lotion and sunscreen formulation may include:

| | | | | PER- |
|---|---|---|---|---|
| | PERCENT- | | DENSITY | CENT |
| | AGE | DENSITY | X % | RANGE |
| ACTIVE | | | | |
| INGREDIENTS | | | | |
| Soybean Oil | 31.5 | 0.915 | 29.344 | PLUS- |
| | | | | MINUS |
| | | | | 15% |
| Citronella Oil | 2.4 | 0.855 | 2.052 | PLUS- |
| | | | | MINUS |
| | | | | 30% |
| Peppermint Oil | 1 | 0.905 | 0.905 | PLUS- |
| | | | | MINUS |
| | | | | 30% |
| INERT | | | | |
| INGREDIENTS | | | | |
| Sunflower Oil | 36 | 0.924 | 33.264 | PLUS- |
| | | | | MINUS |
| | | | | 40% |
| Beeswax | 10 | 0.96 | 9.6 | PLUS- |
| | | | | MINUS |
| | | | | 30% |
| Zinc Oxide | 6 | 5.61 | 33.66 | PLUS- |
| (6%) | | | | MINUS |
| | | | | 45% |
| Isopropyl | 4.4 | 0.7854 | 3.45576 | PLUS- |
| Alcohol | | | | MINUS |
| | | | | 20% |
| Water | 5 | 1 | 5 | PLUS- |
| | | | | MINUS |
| | | | | 35% |
| Vanillin | 2 | 1.056 | 2.112 | PLUS- |
| | | | | MINUS |
| | | | | 40% |
| Turmeric Oil | 1.5 | 0.92 | 1.38 | PLUS- |
| | | | | MINUS |
| | | | | 40% |
| Lecithin | 0.2 | 1.03 | 0.206 | PLUS- |
| | | | | MINUS |
| | | | | 25% |

As specified above, the composition may include bee [symbol] s wax, soybean oil, an edible oil such as sunflower oil, isopropyl alcohol, peppermint oil, citronella oil, zinc oxide, water and turmeric oil. The bee [symbol] s wax may be present in an amount of from 5 to 20% by weight. The soybean oil may be present in an amount of from 15 to 40% by weight. The sunflower oil may be present in an amount of from 30 to 50% by weight. The isopropyl alcohol may be present from 3 to 15% by weight. The peppermint oil may be present at about 1% by weight. The citronella oil may also be present in an amount of from 1% to 5% by weight. The turmeric oil may be present at about 1.5% by weight. Water may be present in an amount of from 2 to 10% by weight. Zinc oxide may be present in an amount of 3 to 10% by weight. Additionally, the composition may include powdered ingredients such as vanillin present at about 2% by weight and Lecithin present at about 0.2% by weight. The weight percentages are based on a total weight of the composition.

The active ingredients in the above formulation may include: soybean oil, citronella oil, and peppermint oil. The bee [symbol] s wax, sunflower oil, isopropyl alcohol, zinc oxide, water, vanillin, lecithin and turmeric oil may be inert ingredients.

The composition of example 3 was made according to the following procedure. The soybean oil and sunflower oils were heated from room temperature to 145 to 160 degrees F. in a mixing vat under agitation. The vanillin, lecithin and bee [symbol] s wax were added to the composition when the temperature approaches 100 F. The mixture is heated and agitated until the mixture clears. Once the mixture clears heat is removed from the composition. At Temperature of 145 F the zinc oxide is added and shearing of the mixture begins. Water is then added to the composition under agitation and the composition is cooled to 110 F. Isopropyl alcohol is added at the temperature of 110 F. The mixture immediately cools to 100 F. At this time the peppermint oil, citronella oil and turmeric oil are added to the composition. The composition is agitated and sheared for 30 minutes. After which time the lotion may be packaged.

### Example 4

In a further aspect, the lotion and sunscreen formulation may include:

| | | | | PER- |
|---|---|---|---|---|
| | PERCENT- | | DENSITY | CENT |
| | AGE | DENSITY | X % | RANGE |
| ACTIVE | | | | |
| INGREDIENTS | | | | |
| Soybean Oil | 30 | 0.915 | 29.344 | PLUS- |
| | | | | MINUS |
| | | | | 15% |
| Citronella Oil | 3 | 0.855 | 2.565 | PLUS- |
| | | | | MINUS |
| | | | | 30% |
| Peppermint Oil | 2 | 0.905 | 0.905 | PLUS- |
| | | | | MINUS |
| | | | | 30% |
| Cinnamon Oil | 2 | 1.04 | 2.08 | PLUS- |
| | | | | MINUS |
| | | | | 30% |
| INERT | | | | |
| INGREDIENTS | | | | |
| Sunflower Oil | 32.15 | 0.924 | 29.7066 | PLUS- |
| | | | | MINUS |
| | | | | 40% |
| Beeswax | 10.5 | 0.96 | 10.08 | PLUS- |
| | | | | MINUS |
| | | | | 30% |
| Triethyl citrate | 5 | 1.136 | 5.68 | PLUS- |
| | | | | MINUS |
| | | | | 15% |
| Salt | 0.15 | 2.16 | 0.324 | PLUS- |
| | | | | MINUS |
| | | | | 20% |
| Zinc Oxide | 6 | 5.61 | 33.66 | PLUS- |
| (6%) | | | | MINUS |
| | | | | 45% |
| Isopropyl | 2 | 0.7854 | 1.5708 | PLUS- |
| Alcohol | | | | MINUS |
| | | | | 20% |
| Water | 2 | 1 | 2 | PLUS- |
| | | | | MINUS |
| | | | | 35% |
| Vanillin | 5 | 1.056 | 5.28 | PLUS- |
| | | | | MINUS |
| | | | | 40% |
| Lecithin | 0.2 | 1.03 | 0.206 | PLUS- |
| | | | | MINUS |
| | | | | 25% |

As specified above, the composition may include bee [symbol] s wax, soybean oil, an edible oil such as sunflower oil, isopropyl alcohol, peppermint oil, citronella oil, cinnamon oil, zinc oxide, triethyl citrate, salt, water and turmeric oil. The bee [symbol] s wax may be present in an amount of from 7 to 13% by weight. The soybean oil may be present in an amount of from 12 to 25% by weight. The sunflower oil may be present in an amount of from 13 to 30% by weight. The isopropyl alcohol may be present from 0.5 to 1.5% by weight. The citronella oil may be present in an amount of from 4% to 5% by weight. The peppermint oil may be present in an amount of from 1.5% to 2.5% by weight. The cinnamon oil may be present in an amount of from 1.5% to 2.5% by weight. The triethyl citrate may be present in an amount of from 1.5 to 7% by weight. Water may be present in an amount of from 2 to 10% by weight. Zinc oxide may be present in an amount of 3 to 10% by weight. Additionally, the composition may include powdered ingredients such as vanillin present in an amount of from 2 to 6% by weight and Lecithin present at about 0.2% by weight. Salt may be present in an amount of from 0.13 to 0.18% by weight. The weight percentages are based on a total weight of the composition.

The active ingredients in the above formulation may include: soybean oil, citronella oil, cinnamon oil and peppermint oil. The bee [symbol] s wax, sunflower oil, isopropyl alcohol, zinc oxide, water, salt, triethyl citrate, vanillin, lecithin and turmeric oil may be inert ingredients.

The composition of example 4 was made according to the following procedure. The soybean oil and sunflower oils were heated from room temperature to 145 to 160 degrees F. in a mixing vat under agitation. The vanillin, lecithin and bee [symbol] s wax were added to the composition when the temperature approaches 100 F. The mixture is heated and agitated until the mixture clears. Once the mixture clears heat is removed from the composition. At Temperature of 145 F the zinc oxide is added and shearing of the mixture begins. Water is then added to the composition under agitation and the composition is cooled to 110 F. Isopropyl alcohol is added at the temperature of 110 F. The mixture immediately cools to 100 F. At this time the peppermint oil, citronella oil, cinnamon oil, triethyl citrate, salt and turmeric oil are added to the composition. The composition is agitated and sheared for 30 minutes. After which time the lotion may be packaged.

### Example 5

In a further aspect, the lotion and sunscreen formulation may include:

| | | | | PERCENT |
|---|---|---|---|---|
| | PERCENTAGE | DENSITY | densityx % | RANGE |
| ACTIVE INGREDIENTS | | | | |
| Soybean Oil | 22.4 | 0.915 | 20.496 | PLUS- |
| | | | | MINUS |
| | | | | 15% |
| Geraniol | 4.5 | 0.889 | 4.0005 | PLUS- |
| | | | | MINUS |
| | | | | 30% |
| Clove Oil | 2 | 0.952 | 1.904 | PLUS- |
| | | | | MINUS |
| | | | | 30% |
| Thyme Oil | 1 | 0.941 | 0.941 | PLUS- |
| | | | | MINUS |
| | | | | 30% |
| INERT INGREDIENTS | | | | |
| Sunflower Oil | 21 | 0.924 | 19.404 | PLUS- |
| | | | | MINUS |
| | | | | 40% |
| Coconut oil | 16 | 0.95 | 15.2 | PLUS- |
| | | | | MINUS |
| | | | | 50% |
| Beeswax | 10 | 0.96 | 9.6 | PLUS- |
| | | | | MINUS |
| | | | | 30% |
| Zinc Oxide | 12 | 5.61 | 39.27 | PLUS- |
| | | | | MINUS |
| | | | | 45% |
| Water | 4.65 | 1 | 4.65 | PLUS- |
| | | | | MINUS |
| | | | | 35% |
| Steric Acid/Cetyl Esters | 3 | 0.941 | 2.823 | PLUS- |
| | | | | MINUS |
| | | | | 50% |
| Vanillin | 4.5 | 1.056 | 4.752 | PLUS- |
| | | | | MINUS |
| | | | | 40% |
| Triethyl Citrate | 2.75 | 1.137 | 3.12675 | PLUS- |
| | | | | MINUS |
| | | | | 15% |
| Isopropyl Alcohol | 1 | 0.7854 | 0.7854 | PLUS- |
| | | | | MINUS |
| | | | | 20% |
| Lecithin | 0.2 | 1.03 | 0.206 | PLUS- |
| | | | | MINUS |
| | | | | 25% |

As specified above, the composition may include bee [symbol] s wax, soybean oil, an edible oil such as sunflower oil and/or Coconut oil, isopropyl alcohol, clove oil, thyme oil, geraniol, zinc oxide, triethyl citrate, water, and Steric Acid/Cetyl Esters.

The bee [symbol] s wax may be present in an amount of from 7 to 13% by weight. The soybean oil may be present in an amount of from 12 to 25% by weight. The sunflower oil may be present in an amount of from 13 to 30% by weight. The isopropyl alcohol may be present from 0.5 to 1.5% by weight. The geraniol may be present in an amount of from 4% to 5% by weight. The clove oil may be present in an amount of from 1.5% to 2.5% by weight. The thyme oil may also be present in an amount of from 0.5% to 1.5% by weight. The Coconut oil may be present in an amount of from 8 to 34% by weight. The Steric Acid/Cetyl Esters may be present in an amount of from 1.5 to 4.5% by weight. The triethyl citrate may be present in an amount of from 2.3 to 3.1% by weight. Water may be present in an amount of from 3 to 5.2% by weight. Zinc oxide may be present in an amount of 4 to 10% by weight. Additionally, the composition may include powdered ingredients such as vanillin present in an amount of from 2 to 6% by weight and Lecithin present at about 0.2% by weight. The weight percentages are based on a total weight of the composition.

The active ingredients in the above formulation may include: soybean oil, clove oil, thyme oil and geraniol. The bee [symbol] s wax, sunflower oil, 50/50 MCT-coconut oil, isopropyl alcohol, zinc oxide, water, Steric Acid/Cetyl Esters, triethyl citrate, vanillin, and lecithin may be inert ingredients.

The composition of example 5 was made according to the following procedure. The soybean oil and sunflower oils were heated from room temperature to 145 to 160 degrees F. in a mixing vat under agitation. The lecithin and water were added to the composition when the temperature approaches 100 F. The mixture is heated and agitated until about 125 degrees F. and a shearing mechanism is activated and maintained until the mixture contains no grit. Next, Steric Acid/Cetyl Esters, triethyl citrate, vanillin and MCT-coconut oil are added to the composition under agitation. Next the bee [symbol] s wax is added at temperature of 145 F and again the shearing mechanism is activated and maintained until the mixture contains no grit. As the shearing continues, the zinc oxide is added slowly to the composition.

The composition is maintained under agitation and heat is tuned off from the composition. Isopropyl alcohol, clove oil, thyme oil and geraniol are added and the composition cools to about 125-130 F. The composition is agitated and sheared for a specified time dependent upon the size of the batch. After which time the lotion may be packaged.

### Example 6

In one aspect, the lotion formulation may include a sunscreen component with a SPF value of 30. The formulation may include:

| | PERCENTAGE | RANGE |
|---|---|---|
| ACTIVE INGREDIENTS | | |
| Soybean Oil | 22.4 | 19 to 26% |
| Geraniol | 4.5 | 3 to 6% |
| Clove Oil | 2 | 1.5 to 3% |
| Thyme Oil | 1 | 0.5 to 1.5% |
| INERT INGREDIENTS | | |
| Sunflower Oil | 19 | 12 to 29 |
| Beeswax | 4 | 3 to 5% |
| Zinc Oxide | 16 | 10 to 20% |
| Water | 21.5 | 15-30% |
| Steric Acid | 1.5 | 1 to 3% |
| Vanillin | 4.4 | 3 to 6% |
| Triethyl Citrate | 3 | 2.5 to 3.5% |
| Lecithin | 0.7 | 0.5 to 1% |

As specified above, the composition may include: soybean oil, clove oil, thyme oil, geraniol, water, an edible oil such as sunflower oil, zinc oxide, vanillin, bee [symbol] s wax, steric acid, triethyl citrate and lecithin.

The soybean oil may be present in an amount of from 19 to 26% by weight. The geraniol may be present in an amount of from 3% to 6% by weight. The clove oil may be present in an amount of from 1.5% to 3% by weight. The thyme oil may also be present in an amount of from 0.5% to 1.5% by weight. Water may be present in an amount of from 15 to 30% by weight. The sunflower oil may be present in an amount of from 12 to 29% by weight. Zinc oxide may be present in an amount of 10 to 20% by weight. The bee [symbol] s wax may be present in an amount of from 3 to 5% by weight. The Steric Acid may be present in an amount of from 1 to 3% by weight. The triethyl citrate may be present in an amount of from 2.5 to 3.5% by weight. Additionally, the composition may include powdered ingredients such as vanillin present in an amount of from 3 to 6% by weight and Lecithin present from 0.5 to 1% by weight. The weight percentages are based on a total weight of the composition.

The active ingredients in the above formulation may include: soybean oil, clove oil, thyme oil and geraniol. The bee [symbol] s wax, sunflower oil, zinc oxide, water, Steric Acid, triethyl citrate, vanillin, and lecithin may be inert ingredients.

In one aspect, the zinc oxide may include a mixture of different sized particles. The mixture may include a half zinc oxide particles having a size from 20-50 μm and half having a size of from 80-200 μm.

The composition of example 6 was made according to the following procedure. The soybean oil and sunflower oils were heated from room temperature to 145 to 160 degrees F. in a mixing vat under agitation. The lecithin and water were added to the composition when the temperature approaches 100 F. The mixture is heated and agitated until about 125 degrees F. and a shearing mechanism is activated and maintained until the mixture contains no grit. Next, Steric Acid, triethyl citrate, vanillin are added to the composition under agitation. Next the bee [symbol] s wax is added at temperature of 145 F and again the shearing mechanism is activated and maintained until the mixture contains no grit. As the shearing continues, the zinc oxide is added slowly to the composition.

The composition is maintained under agitation and heat is tuned off from the composition. Clove oil, thyme oil and geraniol are added and the composition cools to about 125-130 F. The composition is agitated and sheared for a specified time dependent upon the size of the batch. After which time the lotion may be packaged.

Testing

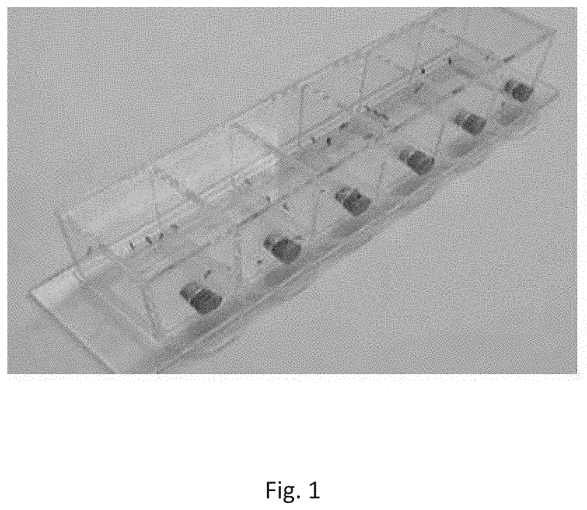

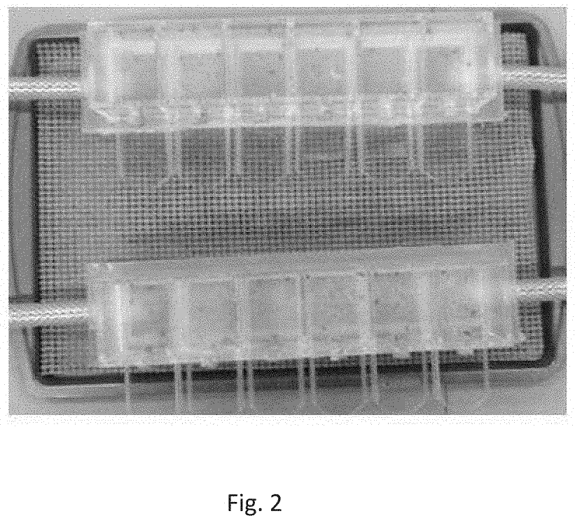

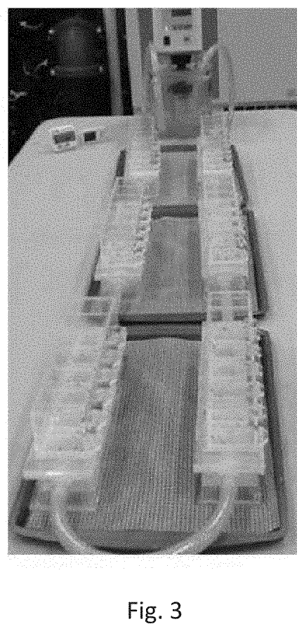

The deterrent effect of the above-described compositions of examples 1 and 2 was tested in a controlled environment. In vitro laboratory tests were conducted and measured and compared to DEET and nontreated control groups for yellow fever mosquitos, Aedes aegypti. Bioassays were conducted in nine six-chambered Plexiglas K & D modules stocked with female Aedes aegypti mosquitos as shown in FIGS. 1-3. Five female mosquitos were aspirated into each of the six chambers of the nine modules. Repellant treatments were applied at 26.7 microliters with a pipette and spread with a small brush over 12 square centimeter sections of organdy cloth attached to a flat plastic template containing rectangular openings. The treated cloth covered template was fitted between the K & D module and a lower Plexiglas unit. The lower Plexiglas unit contained shallow surface wells filled with a blood substitute (CDTA and ATP) and covered with a moistened collagen membrane. The blood substitute was heated to 38° C. using water pumped through hose lines attached to the lower units and a water bath supplied with a temperature controlled inversion circulator. Mosquitos were exposed to the treated surfaces by opening sliding doors beneath the modules for 90-second biting counts at five post-treatment time intervals from 1 to 6 hours. Fresh, untreated mosquitos are used for each time interval.

The bite counts were tabulated and utilized for statistical analysis. The percent repellency of the formulations as described above was more effective than DEET over the five time periods. At the initial start time of zero the repellency of the composition as described herein was 100% while DEET was 91.9%. The average for all five time periods demonstrates the repellency of 94.54% for the compositions as described herein compared to 86.52% for DEET. At the 6 hour time period the compositions as described herein included a repellency of 96.8% effective compared to DEET which was 87.1% effective.

The testing protocol including the in vitro environment provides a rigorous testing of the repellency of the composition as the mosquitos are confined with a blood meal.

An unpaired t-test was conducted on the biting counts between lotion as described herein and the control group where there was no treatment applied between the mosquito and the blood well. The results of this test showed highly significant results between the mean biting counts of 0.33 for the compositions as described herein compared to 3.44 for the control group. The statistical factors include t=5.8704 where df=16 and a standard error of difference is equal to 0.530. The two-tailed p value is less than 0.0001. The reduction in biting pressure when using the compositions compared to the control group is significant at the 99.99 level of significance.

The experimental results demonstrate a very high effectiveness of repellency utilizing a safe and effective organic product that is both DEET and chemical free. The testing has demonstrated that the product is effective in repelling mosquitos and in particular the yellow fever mosquito which may potentially carry West Nile virus.

Additional testing was performed with the lotion of example 3 including sunflower oil, isopropyl alcohol, peppermint oil, citronella oil, zinc oxide, water and turmeric oil with the following protocols. Aedes aegypti mosquitoes were obtained from insecticide-susceptible reference strains held at the London School of Hygiene & Tropical Medicine, UK. All mosquitoes are reared and housed under optimal environmental conditions of 25° C.±2° C. and 80% Relative Humidity with a 12:12 hour photoperiod. Testing was carried out in a testing room maintained at 25±2K and 80% RH with a 12:12 hour photoperiod. For each product, the same person carried out the arm-in-cage testing.

For each product test, three batches of 50 female mosquitoes were placed in cages 30×30×30 cm inside the testing room. Before the start of each test a bare arm with gloved hand was inserted into the cage for 30 seconds to assess the biting readiness of the mosquitoes. Only cages with at least ten mosquitoes landing within 30 seconds were used in subsequent testing. The number of mosquitoes probing the untreated arm was counted at the end of the 30 second test.

Following the combined fitness/control test, the product was applied onto the right arm at the WHO standard rate of 1 ml per 600 cm2 (1.6711 per crn2) for DEET, and at a rate of 2 mg per cm′ for the Sunscreen Repellent. The arm was then inserted into the cage for 30 seconds and the number of mosquitoes probing on the arm counted and recorded. The procedure was repeated with a total of three cages. This was repeated for each of the three cages and at hourly intervals until 8 hours post-application or until protection drops below 50%. Each product was tested on a separate test day.

Protective efficacy (PE) was calculated using the following formula: PE=(Mosquitoes probing on control arm [symbol] Mosquitoes probing on treated arm)/Mosquitoes probing on control arm×100.

The average probing on the control arm during testing was 17 and 21 mosquitoes at the end of the 30 second exposure time, in cages used for testing the Sunscreen Repellent Lotion and 8% DEET respectively. The Sunscreen Repellent Lotion provided 100% protection at 0 hours, whereas the 8% DEET provided 100% protection for 1 hour after application (Table 1). The Sunscreen Repellent Lotion provided 50% protection for 2 hours.

TABLE 1
Total Protective Efficacy of the Sunscreen Repellent
Lotion and 8% DEET against Aedes aegypti mosquitos

| | | | No. | No. | | |
|---|---|---|---|---|---|---|
| | | | probing | probing | | |
| | | | on bare | on | | |
| Hours after | Time | | control | treated | | Total |
| Application | (hh:mm) | Replicate | arm | arm | % PE | % PE |
| 0 | 11:24 | 1 | 17 | 0 | 100.00 | 100.00 |
| | 11:27 | 2 | 22 | 0 | 100.00 | |
| | 11:29 | 3 | 14 | 0 | 100.00 | |
| 1 | 12:26 | 1 | 19 | 1 | 94.74 | 94.12 |
| | 12:28 | 2 | 20 | 2 | 90.00 | |
| | 12:30 | 3 | 12 | 0 | 100.00 | |
| 2 | 13:25 | 1 | 25 | 2 | 96.00 | 92.98 |
| | 13:28 | 2 | 22 | 1 | 95.45 | |
| | 13:30 | 3 | 10 | 2 | 80.00 | |
| 3 | 14:30 | 1 | 19 | 14 | 26.32 | 37.50 |
| | 14:32 | 2 | 11 | 6 | 45.45 | |
| | 14:34 | 3 | 10 | 5 | 50.00 | |

COMPARATIVE TABLE 2
Total Protective Efficacy of 8% DEET against Aedes aegypti

| | | | No. | No. | | |
|---|---|---|---|---|---|---|
| | | | probing | probing | | |
| | | | on bare | on | | |
| Hours after | Time | | control | treated | | Total |
| Application | (hh:mm) | Replicate | arm | arm | % PE | % PE |
| 0 | 09:40 | 1 | 16 | 0 | 100.00 | 100.00 |
| | 09:42 | 2 | 28 | 0 | 100.00 | |
| | 09:44 | 3 | 30 | 0 | 100.00 | |
| 1 | 10:40 | 1 | 15 | 0 | 100.00 | 100.00 |
| | 10:42 | 2 | 23 | 0 | 100.00 | |
| | 10:43 | 3 | 35 | 0 | 100.00 | |
| 2 | 11:42 | 1 | 12 | 1 | 91.67 | 95.00 |
| | 11:44 | 2 | 22 | 2 | 90.91 | |
| | 11:46 | 3 | 26 | 0 | 100.00 | |
| 3 | 12:40 | 1 | ND | ND | — | 95.35 |
| | 12:42 | 2 | 20 | 2 | 90.00 | |
| | 12:46 | 3 | 23 | 0 | 100.00 | |
| 4 | 01:40 | 1 | 24 | 2 | 91.67 | 95.16 |
| | 01:42 | 2 | 17 | 1 | 94.12 | |
| | 01:44 | 3 | 21 | 0 | 100.00 | |
| 5 | 02:40 | 1 | 29 | 2 | 93.10 | 90.00 |
| | 02.42 | 2 | 18 | 2 | 88.89 | |
| | 02:44 | 3 | 23 | 3 | 86.96 | |
| 6 | 03:40 | 1 | 17 | 10 | 41.18 | 54.90 |
| | 03:42 | 2 | 14 | 6 | 57.14 | |
| | 03:44 | 3 | 20 | 7 | 65.00 | |
| 7 | 04:40 | 1 | 11 | 7 | 36.36 | 46.34 |
| | 04:42 | 2 | 12 | 6 | 50.00 | |
| | 04:44 | 3 | 18 | 9 | 50.00 | |

The sunscreen lotion showed complete protective efficacy (0 hours) and 50% protective efficacy at 2 hours. DEET (8%) provided the longer complete protective efficacy (1 hour) and 50% protective efficacy time (6 hours).

SPF Testing

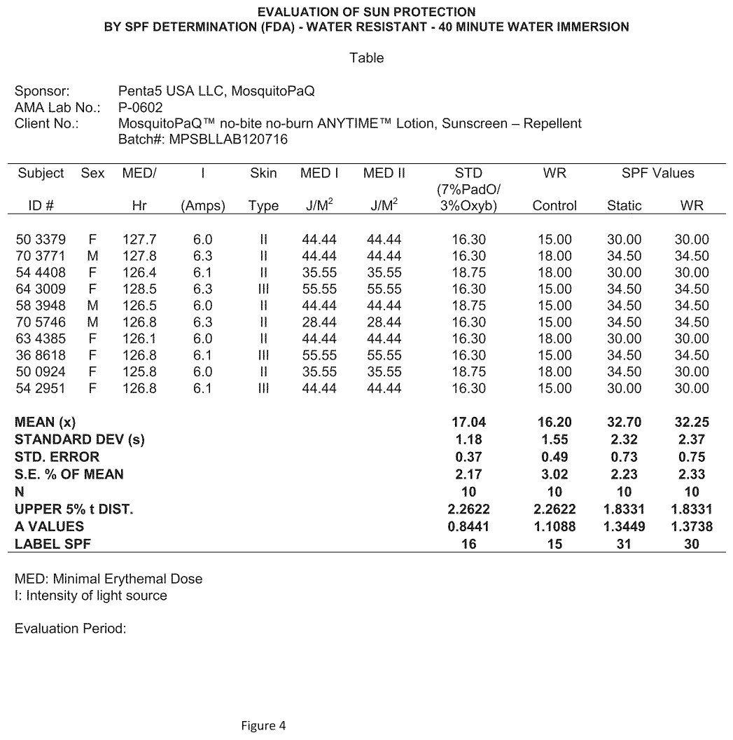

Referring to FIG. 4 there is shown a detailed report regarding the SPF of the composition of Examples 1-3. The compositions were subjected to a 40 minute water immersion test. The report indicates a SPF of 30+ for the compositions.

Additional Testing

Additional field testing of the sunscreen lotion formulations was performed.

Sarasota, Fla.

Several visits to beaches in Sarasota, Fla. during the summer months of 2016 with application of the compositions of examples 1-3 applied to the skin of a person. It was observed:

a) No mosquito bites during stays as long as 4 hours [symbol] repellency of 100%, b) No reduction in repellency after trips into Gulf waters during the 4 hour+ stays c) Nearby beach goers and swimmers were experiencing bites from mosquitoes.

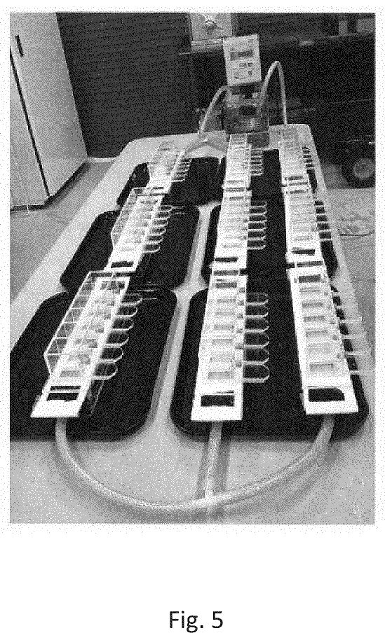

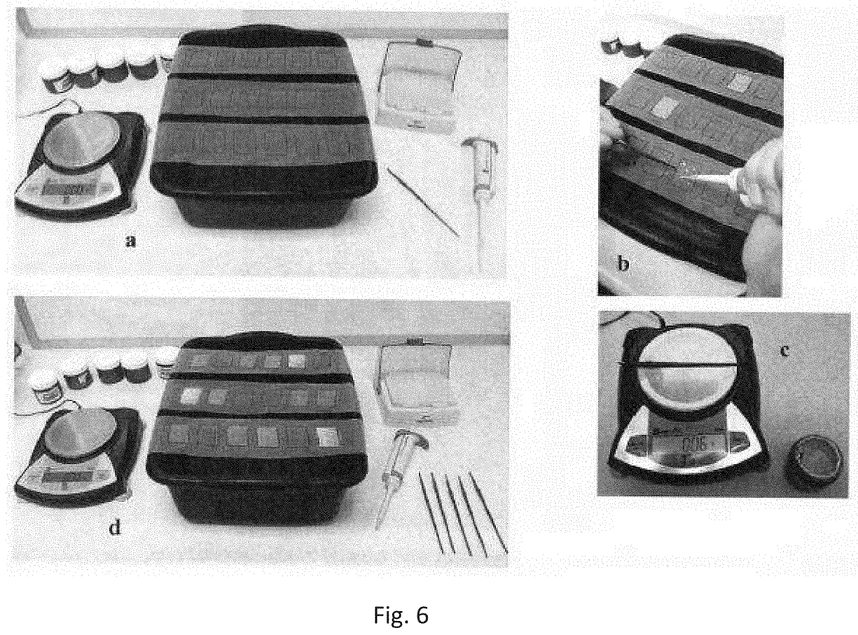

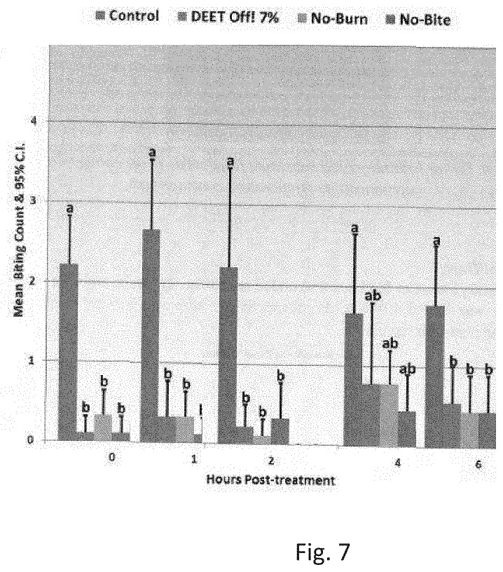

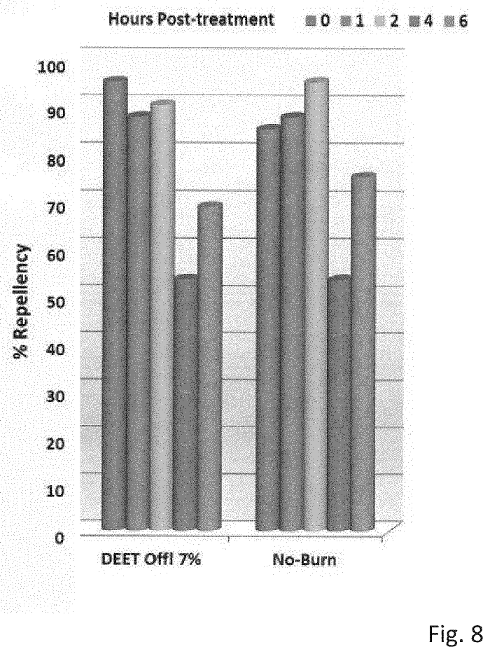

The deterrent effect of the above-described compositions of example 4 (represented as NO-Burn in FIGS. 8-9) was tested in a controlled environment. In vitro laboratory tests were conducted and measured and compared to DEET and nontreated control groups for yellow fever mosquitos, Aedes aegypti. The repellency of the formulations were compared to a DEET standard, and a non-treated control against yellow fever mosquitoes, Aedes aegypti. The examples were performed in a temperature-controlled laboratory following techniques as disclosed in Klun, L. A., M. A. Kramer, A. Zhang, S. Wang, and M. Debboun. 2008. A quantitative in vitro assay for mosquito deterrent activity without human blood cells. J Am. Mosq. Contr. Assoc. 24:508-512. Bioassays were conducted in nine, six-chambered Plexiglasss K&D modules interconnected with hoses to a water bath supplied with a temperature-controlled inversion circulator as displayed in (FIG. 5). Five colony-reared female Aedes aegypti were aspirated into each chamber (FIG. 1). Nine replications of the following treatments were randomly assigned to the chambers: No-Bite; No-Burn; Positive control—7% DEET standard (Off! Family Care Unscented) and Negative control—Non-treated. Repellents were applied to nine ca. 2″×2″×9″ strips of organdy cloth and spread with a small paint brush over 12 cm′ ink-pen-demarcated rectangles drawn on the strips with a flat plastic template. A micropipette set at 27.6 μ1 was used to apply liquid repellents, while creams were applied at 0.06 g (FIG. 6 a-d). Treated clothes were taped to flat plastic templates with openings aligning with lower Plexiglasss bases. The lower Plexiglasss bases contained shallow surface wells that were filled with a blood substitute (CDTA and ATP) and covered with moistened collagen membranes. The blood substitute was heated to 38° C. with water pumped through hose lines attached to the lower base and the water bath. The plastic templates were fitted between the K&D modules and lower Plexiglasss bases (FIG. 5). Mosquitoes were exposed to the treated surfaces by opening the K&D module sliding doors for 90-second biting counts at five post-treatment time intervals (0, 1, 2, 4 & 6 hrs) (FIG. 1). Fresh mosquitoes were aspirated into the chambers for each time interval. Ambient temperature and humidity were continuously recorded throughout the study with a HOBO data logger as displayed in FIG. 7. The dependent variable was biting count mean. Treatment and module means were independent variables. Biting counts as well as log and square root transformed biting counts by treatment were examined for normality with SAS PC Proc Univariate. The variance in biting count means by treatment and time intervals was statistically analyzed with SAS Proc ANOVA and statistically tested for significant differences with Tukey's Studentized Range test at p<0.05 and charted with 95% Confidence Intervals (C.I.). Average % repellency was calculated with the following formula and charted by treatment and time interval: (Control Avg. Biting Count−Treatment Avg. Biting Count)÷Control Avg. Biting Count×100.

Normality Tests:

Biting counts by treatment were found to follow closer to a normal distribution compared to log or square root transformed data. Consequently, all statistical analysis was conducted on non-transformed data.

Biting Count and Repellency:

There was no significant difference (p>0.05) in average biting counts among the nine K&D modules. Biting counts in the controls were significantly (p<0.05) greater than all of the repellent treatments at 0, 1, 2, and 6 hours post-treatment (FIG. 8). At 4-hours, there was no significance difference (p>0.05) in biting activity among the control, DEET and no-burn/no-bite. There was no significant difference (p>0.05) in bites among the repellents at 4 hours. When biting count data were converted to percent repellency, all of the formulations appeared to perform as well, if not better, than the 7% DEET (FIG. 9).

Testing

The deterrent effect of the above-described compositions (represented as NO-Burn in FIGS. 7-8) was tested in a controlled environment. In vitro laboratory tests were conducted and measured and compared to DEET and nontreated control groups for yellow fever mosquitos, Aedes aegypti. The repellency of the formulations were compared to a DEET standard, and a non-treated control against yellow fever mosquitoes, Aedes aegypti. The examples were performed in a temperature-controlled laboratory following techniques as disclosed in Klun, J. A., M. A. Kramer, A. Zhang, S. Wang, and M. Debboun. 2008. A quantitative in vitro assay for mosquito deterrent activity without human blood cells. J Am. Mosq. Contr. Assoc. 24:508-512. Bioassays were conducted in nine, six-chambered Plexiglasss K&D modules interconnected with hoses to a water bath supplied with a temperature-controlled inversion circulator as displayed in (FIG. 5). Five colony-reared female Aedes aegypti were aspirated into each chamber (FIG. 1). Nine replications of the following treatments were randomly assigned to the chambers: No-Bite; No-Burn; Positive control—7% DEET standard (Off! Family Care Unscented) and Negative control—Non-treated. Repellents were applied to nine ca. 2″×2″×9″ strips of organdy cloth and spread with a small paint brush over 12 cm′ ink-pen-demarcated rectangles drawn on the strips with a flat plastic template. A micropipette set at 27.6 μ1 was used to apply liquid repellents, while creams were applied at 0.06 g (FIG. 6 a-d). Treated clothes were taped to flat plastic templates with openings aligning with lower Plexiglasss bases. The lower Plexiglasss bases contained shallow surface wells that were filled with a blood substitute (CDTA and ATP) and covered with moistened collagen membranes. The blood substitute was heated to 38° C. with water pumped through hose lines attached to the lower base and the water bath. The plastic templates were fitted between the K&D modules and lower Plexiglasss bases (FIG. 4). Mosquitoes were exposed to the treated surfaces by opening the K&D module sliding doors for 90-second biting counts at five post-treatment time intervals (0, 1, 2, 4 & 6 hrs) (FIG. 1). Fresh mosquitoes were aspirated into the chambers for each time interval. The dependent variable was biting count mean. Treatment and module means were independent variables. Biting counts as well as log and square root transformed biting counts by treatment were examined for normality with SAS PC Proc Univariate. The variance in biting count means by treatment and time intervals was statistically analyzed with SAS Proc ANOVA and statistically tested for significant differences with Tukey's Studentized Range test at p<0.05 and charted with 95% Confidence Intervals (C.I). Average % repellency was calculated with the following formula and charted by treatment and time interval: (Control Avg. Biting Count−Treatment Avg. Biting Count)÷Control Avg. Biting Count×100.

Normality Tests:

Biting counts by treatment were found to follow closer to a normal distribution compared to log or square root transformed data. Consequently, all statistical analysis was conducted on non-transformed data.

Biting Count and Repellency:

There was no significant difference (p>0.05) in average biting counts among the nine K&D modules. Biting counts in the controls were significantly (p<0.05) greater than all of the repellent treatments at 0, 1, 2, and 6 hours post-treatment (FIG. 7). At 4-hours, there was no significance difference (p>0.05) in biting activity among the control, DEET and no-burn/no-bite. There was no significant difference (p>0.05) in bites among the repellents at 4 hours. When biting count data were converted to percent repellency, all of the formulations appeared to perform as well, if not better, than the 7% DEET (FIG. 8).

Additional Testing

Additional field testing of the sunscreen lotion formulations was performed.

Sarasota, Fla.

Several visits to beaches in Sarasota, Fla. during the summer months of 2016 with application of the compositions of examples 1-3 applied to the skin of a person. It was observed:

a) No mosquito bites during stays as long as 4 hours [symbol] repellency of 100%, b) No reduction in repellency after trips into Gulf waters during the 4 hour+ stays c) Nearby beach goers and swimmers were experiencing bites from mosquitoes.

### Example 6 Testing

The deterrent effect of the above-described compositions of example 6 was tested in a controlled environment.

The efficacy of a natural formulation of a combination of mosquito repellent and sunscreen was assessed by conducting repellency studies using adult mosquitoes reared from larvae sourced from a site in St. Andrew Jamaica, as well as larvae from a susceptible strain of mosquitoes maintained under laboratory conditions. Untreated hands (control) and hands treated with the sample repellants were exposed to the adult mosquitoes and the number of female mosquitoes seeking a blood meal was recorded. The mean number of mosquitoes seeking a blood meal ranged from 10.5±0.76 to 11.3±0.55 for the control compared to 0.50±0.22 for the Example 6 combination. The mean level of repellency was 98.6±1.4% for example 6. The test concluded that both insect repellents were effective against the randomly selected population of mosquitoes.

The study was conducted using mosquito larvae sourced from a site in St. Andrew, as well as larvae from a susceptible strain of mosquitoes maintained under laboratory conditions. Plastic containers (˜1 Liter) containing at least 40 mosquito larvae were fed and placed in specially designed (˜30,000 cm3) testing chambers to allow for pupation and the emergence of adult mosquitoes. The chambers consisted of a PVC frame covered with a 1.2 mm white nylon mesh. At one end was a specially designed opening to allow a human hand to be placed in the chamber while preventing the escape of the mosquitoes.

The chambers were checked several times each day and as soon as a minimum of 20 female adult mosquitoes were observed, the container with the water containing the immature stages was removed in preparation for the assessment. Tests were done using human volunteers. The volunteers [symbol] forearms were washed with water and unscented soap, and then dried. One hand was randomly selected and treated by applying formula of example 6.

The volunteer was asked to place the untreated (control) hand in a test cage containing the adult mosquitoes for a two-minute period and the number of mosquitoes seeking to obtain a blood meal recorded. After two minutes the untreated hand was replaced with the treated hand and a similar assessment done. The untreated hand was again exposed to the adult mosquitoes after the treated hand was removed to assess the mosquitoes continued interest in seeking a blood meal. The assessment was repeated at 10-minute intervals for a total of one hour. Six replicates were done for each treatment.

The percent repellency was determined by calculating the difference between the number of mosquitoes seeking a blood meal from the control hands and the treated hands. The data was collated and analyzed in SPSS Statistics Version 19.

Data on the number of mosquitoes seeking a blood meal from treated and untreated hands are presented in Table 3. The mean number of mosquitoes seeking a blood meal ranged from 10.5±0.76 to 11.3±0.55 for the control compared with 0.17±0.17 to 0.83±0.31 for the treated hands. The mean level of repellency was 98.6±1.4% for the formula of example 6. It should be noted that none of the mosquitoes landing on the treated hands sought to take a blood meal. Based on the data it was concluded that the formula of example 6 was effective in repelling both populations of mosquitoes.

TABLE 3
Number of female mosquitoes from selected mosquito populations seeking
a blood meal from human hands treated with Example 6

| | | | Number of | | |
|---|---|---|---|---|---|
| | | | mosquitoes | | |
| | | St. Andrew | seeking a | Susceptible | |
| | | Strain | blood meal | Strain | Example |
| | Replicates | Control | Example 6 | Control | 6 |
| | 1 | 13 | 0 | 13 | 0 |
| | 2 | 11 | 0 | 10 | 0 |
| | 3 | 9 | 0 | 11 | 1 |
| | 4 | 12 | 1 | 14 | 0 |
| | 5 | 10 | 0 | 14 | 1 |
| | 6 | 8 | 0 | 11 | 1 |
| Mean ± | | 10.5 ± | 0.17 ± | 11.3 ± | 0.50 ± |
| S.E | | 0.76 | 0.17 | 0.55 | 0.22 |

### SPF Testing Example 6

The sample disposition was conducted in compliance with federal, state, and local ordinances. The population demographics of the 50 subjects were as follows:

Sex Male 9

Female 42

Race Caucasian 39

Hispanic 10

Asian 2

Test material is dispensed onto an occlusive, hypoallergenic patch. The patch is applied directly to the skin and the subject is dismissed with instructions not to wet or expose the test area to direct sunlight. The procedure is repeated for nine consecutive 24 hour exposures for Monday, Wednesday, and Friday for 3 consecutive weeks. Comparison is made for 9 inductive responses. At the conclusion the consulting Dermatologist and Pediatrician reviewed the data and confirmed stated conclusions.

The response matrix contains 561 cells and 100% confirm no reaction [symbol] score of ZERO.

Determination of Sunscreen Factor: The procedure for this study is described by the International Standard ISO 24444 [symbol] Cosmetic [symbol] Sun protection test methods. The test material and SPF 15 Standard (P3 Standard) were evenly applied using plastic syringes to rectangular areas measuring a minimum of 30 cm2 for a final concentration of 2.0=±0.05 mg/cm2. The light source simulates the solar UVA and UVB spectrum.

Results of Sun Protection Factor:

The minimum SPF value recorded for Example 6 was SPF=30. The maximum SPF value recorded for example 6 was SPF=33.7.

The minimum SPF value recorded for the 15 SPF control was 15, while the maximum was 18.8.

| | Control 15 SPF | Example 6 |
|---|---|---|
| Mean | 16.9 | 31.5 |
| Standard Dev | 2.0 | 1.9 |
| Std Error | 0.6 | 0.6 |
| 95% Confidence | 15.5-18.3 | 30.1-32.9 |
| N | 10 | 10 |

We claim:

1. A topical mosquito repellent and sunscreen composition comprising:
a lotion including active and inert ingredients, wherein the active ingredients include soybean oil, clove oil, thyme oil and geraniol and the inert ingredients include bee [symbol] s wax, sunflower oil, steric acid, zinc oxide, water, vanillin, lecithin, and triethyl citrate.

2. The topical mosquito repellent composition of claim 1 wherein the bee [symbol] s wax is present in an amount of from 3 to 5% by weight, the soybean oil is present in an amount of from 19 to 26% by weight, the sunflower oil is present in an amount of from 19 to 29% by weight, the geraniol is present in an amount of from 3% to 6% by weight, the clove oil is present in an amount of from 1.5% to 3.0% by weight, the thyme oil is present in an amount of from 0.5% to 1.5% by weight, the Steric Acid is present in an amount of from 1.0 to 3.0% by weight, the triethyl citrate is present in an amount of from 2.5 to 3.5% by weight, water is present in an amount of from 15 to 30% by weight, zinc oxide is present in an amount of 10 to 20% by weight, vanillin is present in an amount of from 3 to 6% by weight and Lecithin is present in an amount of 0.5 to 1.0% by weight,

3. The topical mosquito repellent and sunscreen composition of claim 1 wherein the composition has an SPF of 30 or greater.

4. The topical mosquito repellent and sunscreen composition of claim 1 wherein the zinc oxide comprises a mixture of different sized particles wherein half of the zinc oxide particles have a size from 20-50 μm and half of the zinc oxide particles have a size of from 80-200 μm.

5. A method of repelling insects comprising:
applying a lotion composition to a body and exposing insects to the lotion, the lotion including active and inert ingredients, wherein the active ingredients include soybean oil, clove oil, thyme oil and geraniol and the inert ingredients include bee [symbol] s wax, sunflower oil, steric acid, zinc oxide, water, vanillin, lecithin, and triethyl citrate.

6. The method of claim 5 wherein the bee [symbol] s wax is present in an amount of from 3 to 5% by weight, the soybean oil is present in an amount of from 19 to 26% by weight, the sunflower oil is present in an amount of from 19 to 29% by weight, the geraniol is present in an amount of from 3% to 6% by weight, the clove oil is present in an amount of from 1.5% to 3.0% by weight, the thyme oil is present in an amount of from 0.5% to 1.5% by weight, the Steric Acid is present in an amount of from 1.0 to 3.0% by weight, the triethyl citrate is present in an amount of from 2.5 to 3.5% by weight, water is present in an amount of from 15 to 30% by weight, zinc oxide is present in an amount of 10 to 20% by weight, vanillin is present in an amount of from 3 to 6% by weight and Lecithin is present in an amount of 0.5 to 1.0% by weight.

7. The method of claim 5 wherein the lotion composition has an SPF of 30 or greater.

8. The method of claim 5 wherein the zinc oxide comprises a mixture of different sized particles wherein half of the zinc oxide particles have a size from 20-50 μm and half of the zinc oxide particles have a size of from 80-200 μm.
